(19) United States
(12) Patent Application Publication
(10) Pub. No.: US 2014/0350432 A1
(43) Pub. Date: Nov. 27, 2014

(54) ASSESSMENT OF RELATIVE PROPORTIONS OF ADRENERGIC AND CHOLINERGIC NERVOUS RECEPTORS WITH NON-INVASIVE TESTS

(71) Applicant: lMPETO MEDICAL, Paris (FR)

(72) Inventors: Kamel Khalfallah, Paris (FR); Philippe Brunswick, Paris (FR)

(21) Appl. No.: 14/455,194

(22) Filed: Aug. 8, 2014

Related U.S. Application Data
(63) Continuation-in-part of application No. 13/215,788, filed on Aug. 23, 2011.
(60) Provisional application No. 61/864,178, filed on Aug. 9, 2013.

(51) Int. Cl. A61B 5/00; A61B 5/053

(52) U.S. Cl. CPC A61B 5/4893; A61B 5/0531; A61B 5/4266; A61B 5/7246; A61B 5/4884; A61B 5/7282; A61B 5/742
USPC 600/547

(57) ABSTRACT

A system and method for assessing relative proportions of cholinergic and adrenergic nervous receptors in a patient is disclosed. The system includes: an anode, a cathode, and passive electrode for placement on different regions of the patient body. The method generally includes: applying DC voltage pulses of varying voltage values to stress sweat glands of the patient, collecting data representing the current between the anode and the cathode and the potential of the anode, the cathode, and the passive electrode for each of the different DC voltage, and computing data representing the electrochemical skin conductance of the patient. The computed data representing the electromechanical skin conductance of the patient is reconciled with reference data from control patients having known relative proportions of cholinergic and adrenergic nervous receptors. Thus, the relative proportions of cholinergic and adrenergic nervous receptors in the patient can be determined.

### CROSS-REFERENCE TO RELATED APPLICATIONS

This application is a continuation-in-part (CIP) of U.S. patent application Ser. No. 13/215,788 filed on Aug. 23, 2011, and also claims priority to U.S. Provisional Parent Application No. 61/864,178 filed on Aug. 9, 2013, both of which are incorporated by reference herein in the entirely.

### BACKGROUND OF THE INVENTION

1. Field of the Invention

The invention relates in general to the field of non-invasive medical diagnostic devices and methods in the field of human health. The invention more specifically applies to the field of the assessment of relative proportions of two types of nervous receptors, which are impacted differently by various diseases.

2. Description of the Related Art

The eccrine sweat glands have epithelial cells comprising two kinds of nervous receptors among which the adrenergic receptors, in particular the beta adrenergic receptors, and the cholinergic receptors. Especially on palm of the hands and sole the feet. It has been discovered that the density of these nervous receptors can be impacted by various diseases, and in particular that some diseases can impact the imbalance between these two kinds of nervous receptors.

For instance, Sato et al. have shown in the article “Defective Beta Adrenergic Response of Cystic Fibrosis Sweat Glands in Vivo and In Vitro” (J. Clin. Invest., the American Society for Clinical Investigation, Inc. Volume 73, June 1984, 1763-1773) that Cytic Fibrosis Transmembrane Regulator (CFTR) dysfunction is linked to the beta adrenergic nervous receptors.

This has led to a test for diagnosing cystic fibrosis, developed by Quinton et al. in “Beta-adrenergic Sweat Secretion as a Diagnostic Test for Cystic Fibrosis” (Am J Respir Crit Care Med, Vol. 186, Iss. 8, pp 732-739, Oct. 15, 2012), during which sweat gland potential in response to Beta-adrenergic stimulation was shown to be directly related to the degree of dysregulation of CFTR that regulates chloride transport through chloride channels of the wall of sweat gland ducts.

The decrease in the density of beta adrenergic nervous detectors is therefore a clue for diagnosing cystic fibrosis. It may be also utilized to diagnose diseases and disorders of the nervous system which may be associated with alterations in imbalance between beta-adrenergic and cholinergic sweat glands receptors. Such diseases and conditions may include but are not limited to trauma, infarction, infection, degenerative nerve disease, malignancy, or post-operative changes including but not limited to Alzheimer's Disease, Parkinson's Disease, Huntington's Chorea, and amyotrophic lateral sclerosis. Another application is to use this imbalance between two kind of receptors in sweat glands as the degree of imbalance of nervous autonomic system in periphery and thus as an indicator of cardiac autonomic system (i.e. central) imbalance.

However, the invasive measurement of a density of cholinergic or adrenergic nervous detectors can be cumbersome and tedious for the patient. There is therefore a need for a non-invasive method for assessing a health condition of a patient by monitoring imbalance between nervous receptors.

### SUMMARY OF THE INVENTION

Thus, one object of the present invention is to provide a new non-invasive method for assessing relative proportions of cholinergic and adrenergic nervous receptors. Another object of the invention is to provide a quick and easy method that provides immediate results. Another object of the invention is to allow the monitoring of the evolution of a disease or the treatment of said disease.

According to the invention, a method for assessing relative proportions of adrenergic and cholinergic nervous receptors is provided, the method being performed in a system comprising: an anode and a cathode. The anode and cathode are intended to be placed on different regions of the patient body. The system also includes a plurality of passive electrodes intended to be placed on different regions of the patient body and connected to a mass with high impedances. The system also includes an adjustable DC source, which is controlled in order to feed the anode with a DC current. The method comprising the following steps: applying DC voltage pulses of varying voltage values in order to stress sweat glands of the patient, the voltage pulses lasting given durations allowing the stabilization of electrochemical phenomena in the body in the vicinity of the electrodes, collecting data representative of the current between the anode and the cathode, the potential of the anode, the potential of the cathode, and the potential of at least one passive electrode for the different DC voltages, computing data representative of the electrochemical skin conductance of the patient, reconciling the data representative of the electrochemical skin conductance of the patient with reference data obtained in the same conditions on patients having known relative proportions of cholinergic and adrenergic nervous receptors, and determining relative proportions of cholinergic and adrenergic nervous receptors of the patient.

A system for assessing relative proportions of adrenergic and cholinergic nervous receptors of a patient is also provided, comprising an anode, a cathode and a plurality of passive electrodes intended to be placed on different regions of the patient body. The plurality of passive electrodes are connected to a mass with high impedances. An adjustable DC source is provided, which is controlled in order to feed the anode with pulses of a DC current of varying voltage values for given durations allowing the stabilization of electrochemical phenomena in the body in the vicinity of the electrodes. The system also includes a measuring circuit designed to collect data representative of the current between the anode and the cathode, the potential of the anode, the potential of the cathode, and the potential of at least one of the passive electrodes for the different DC voltages. The system further comprises a computing circuit designed to compute data representative of the electrochemical skin conductance of the patient and to reconcile the data with reference data obtained in the same conditions on patients having known relative proportions of adrenergic and cholinergic nervous receptors.

The system and method according to the invention allow an immediate and non-intrusive method for assessing relative proportions of adrenergic and cholinergic nervous receptors. This proportion allows the system and method to identify the patient as suffering from a disease impacting the density of one of these kinds of nervous receptors. Moreover, a repeated assessment of these relative proportions allows the system and method to monitor the evolution of a disease or to study whether a treatment is effective or not. This allows an earlier detection of evolutionary diseases such as the Parkinson disease and a choice of the most efficient treatment since it can be determined very quickly that one treatment is more efficient than another.

### BRIEF DESCRIPTION OF THE DRAWINGS

The features and advantages of the invention will be apparent from the following more detailed description of certain embodiments of the invention and as illustrated in the accompanying drawings, in which:

FIG. 1 shows a system designed to carry out the method according to the invention;

FIG. 2 shows the main steps carried out in the diagnosis method according to the invention;

FIG. 3 shows the electric diagram of the implementation of the system on a patient body;

FIG. 4a shows an example of electrochemical skin conductance computed respectively for a sound patient and for a patient with impaired densities of nervous receptors;

FIG. 4b shows an example of electrochemical skin conductance computed respectively for a sound patient and for a patient with impaired densities of nervous receptors;

FIG. 5 shows the ratios of electrochemical skin conductance at low and intermediate voltages applied to the skin for control patients and patients with impaired innervation;

FIG. 6a shows a computer system designed to carry out the method according to the invention; and

FIG. 6b shows a plurality of instructions stored in the memory of the computer system illustrated in FIG. 6a.

### DETAILED DESCRIPTION OF A PREFERRED EMBODIMENT

A variation in the density of adrenergic or cholinergic nervous receptors, resulting in a variation of the relative proportions of these two kinds of receptors, results in an altered response of eccrine sweat glands to electric stimulation as illustrated by measurement performed in patients with known CFTR dysfunction as compared to controls.

More particularly, the electrochemical skin conductance of patients varies according to the relative proportions of adrenergic and cholinergic receptors.

This is a basis of the invention, which measures the electrochemical skin conductance (ESC) of a patient after application of a low direct voltage via stainlees-steel or nickel electrodes, and, based on a ratio between values of the electrochemical skin conductance at low and high voltages, infers the relative proportions of adrenergic and cholinergic nervous receptors.

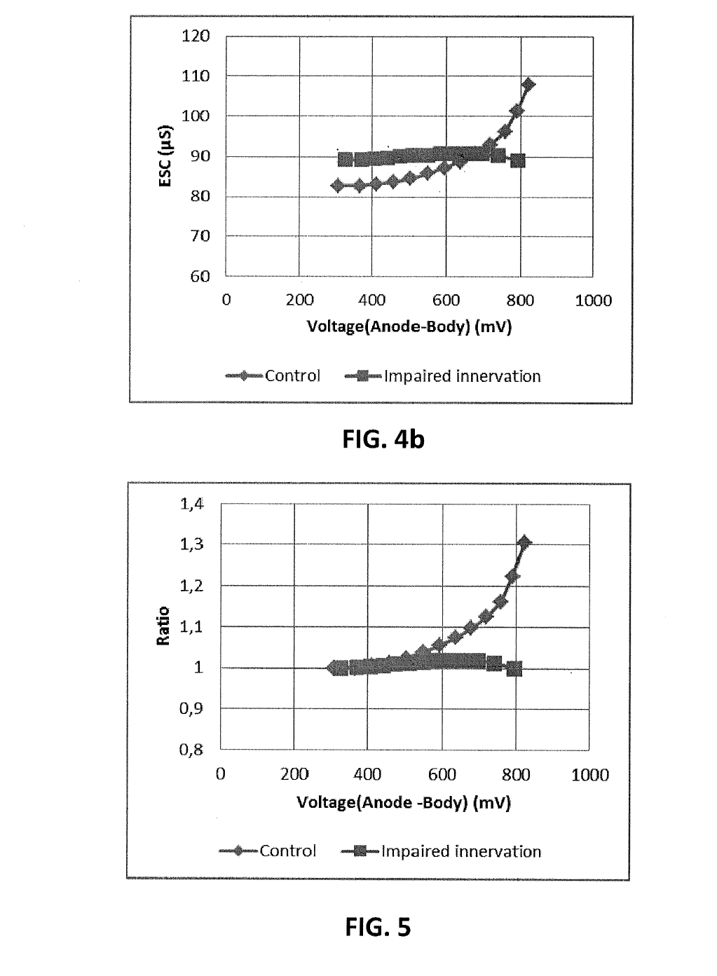

Typical skin responses are shown in FIGS. 4A and 4B for healthy subjects. Above some voltage threshold, the current deviates from linearity and ESC raises. For impaired innervation, the current remains almost linear and ESC is constant.

### Description of a Diagnosis System According to the Invention

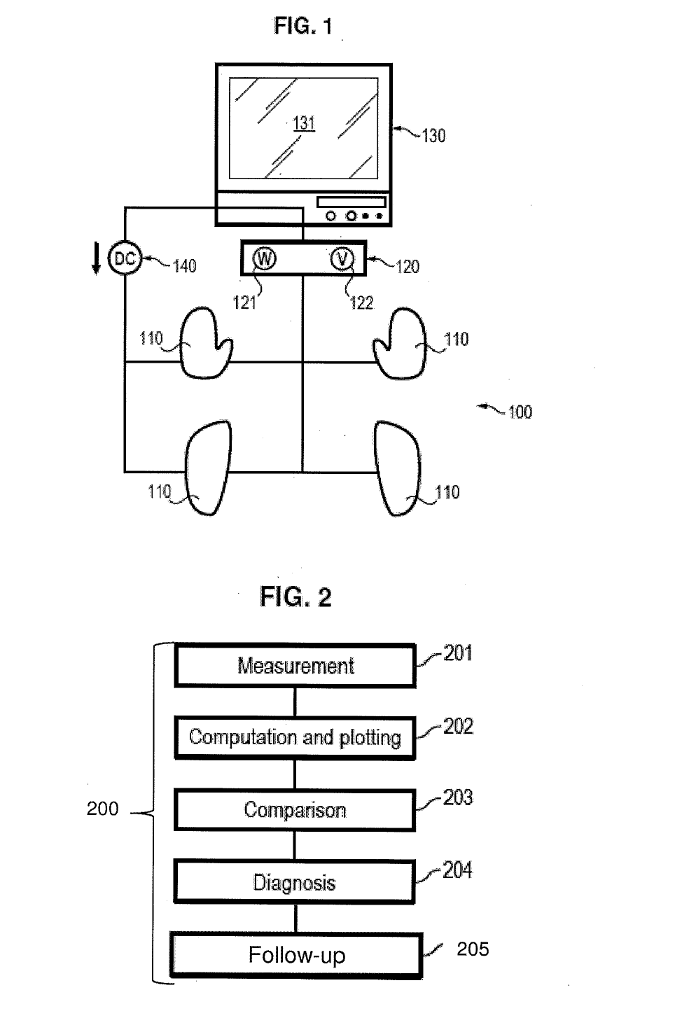

A system 100 for assessing relative proportions of adrenergic and cholinergic nervous receptors of a patient is shown in FIG. 1.

The system 100 comprises a series of large area electrodes 110, preferably four electrodes 110, on which the patient can place his hands and feet. The sites of the electrodes 110 have been chosen because of their high density of eccrine sweat glands.

The electrodes 110 can be made of nickel or stainless steel with sufficient level of nickel. Their individual surface area ranges from 50 cm² to 200 cm², so that they cover substantially all the surface of the hand palms and the feet soles. Yet they can be adapted for children or even infants.

The electrodes 110 are connected to a computer 130 for collecting, computing, and storing data. The electrodes 110 are also connected to an adjustable DC source 140, which is controlled by an operator or the computer 130 to feed the electrodes 110 with a DC current of a determined voltage.

The system 100 also comprises a measuring circuit 120, to measure the voltage potential of each electrode through a voltmeter 122, as well as the current between two of the electrodes 110 through a Wheatstone bridge 121.

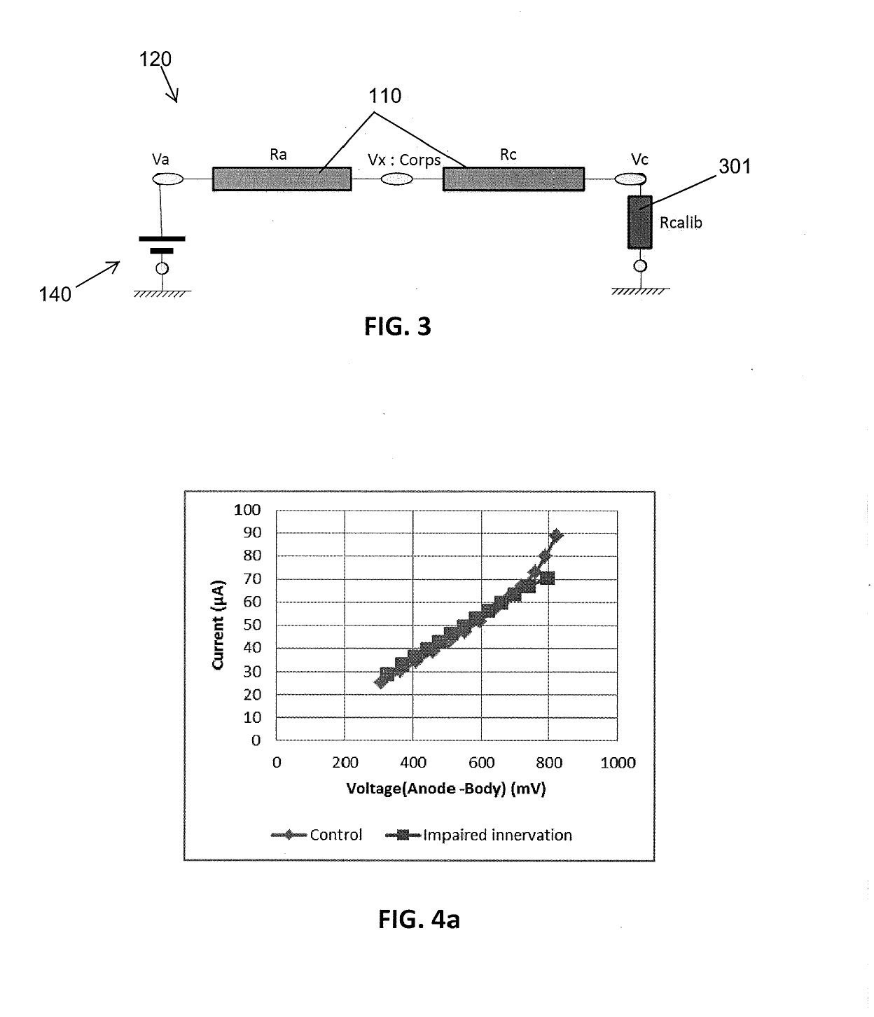

As shown in FIG. 3, the system 100 also comprises a measurement resistor 301, that allows measuring the current flowing through the active electrodes 110 by the measurement of the tension at its terminals.

The system 100 may optionally also comprise another measurement resistor (not shown), disposed between the electric source and the anode, for measuring the current flowing to the anode, and for correcting the current measured at the cathode.

The diagnosing system can also be equipped with a display 131, designed for displaying the measured data as well as the results of the computations carried out on the data.

The method for assessing relative proportions of cholinergic and adrenergic nervous receptors will now be described with reference to FIG. 2.

### Measurement Step 201

In order to assess relative proportions of adrenergic and cholinergic nervous receptors of a patient, the patient places his hands and feet on the large area nickel electrodes 110 and stands up without moving his hands and feet during a 2 minute period when a measurement is taken. The measurement step 201 is carried out independently for the two feet and for the two hands.

For one measurement configuration (for example right-hand, left-hand), one electrode 110 is used as an anode, and another one of the electrodes 110 is used as a cathode. These electrodes 110 may thus be designated as the active electrodes (AE). The potential of the anode is noted Va and the potential of the cathode is noted Vc.

The two other electrode 110 (right-feet, left-feet in the example) are passive. The passive electrodes 110 are connected to a mass with high impedances and allow retrieving the voltage Vx reached by the body (B) by measurement of their potential.

The anode is then fed with DC current. The anode is applied with an initial voltage ranging from 0.5 V to 1.5 V and preferably equal or close to 1 V, during a duration ranging from 0.5 seconds to 2 seconds, and preferably 1 second. The duration must last long enough to allow the stabilization of electrochemical phenomena in the body in the vicinity of the electrodes 110. The applied current induces voltage on the cathode and a current going through the body towards the cathode. The voltage and current of both electrode 110 are measured and stored by the computer 130 at measurement step 201.

Then, the voltage applied to the anode is increased stepwise by a voltage step ranging from 0.1 V to 0.3 V and preferably 0.2 V.

For instance, the voltage applied to the anode may be increased from 1 V up to 1.2 V. This voltage value is applied to the anode at a time with a time interval ranging from 0.5 second to 2 seconds and preferably at 1 second. A new measurement is then performed. Such a progressive step by step increase from 0.1 V to 0.3 V and preferably 0.2 V during preferably a 1 second time interval is applied until a maximal voltage below 10V and preferably from 3.5 V to 4.5 V, and even more preferably around 4 V is reached. This stepwise increase preferably represents a total of 16 measurements, when the minimum voltage value is 1 V, the maximum voltage value is 4 V, and the voltage step is 0.2 V. The following results have been obtained with these experiments conditions.

The same series of measurements can also be carried out in reverse by applying successive pulses of decreasing voltages. The same series of measurements can then be carried out with the electrodes being reversed (anode becoming cathode and vice-versa) and the same can be carried out on the feet.

### Computation and Plotting 202

Once the electrode potentials have been recorded, the electronic board computes the difference in voltages between the active electrodes AE, for instance the anode Va and the body B, noted Δ(AE−B), for each DC voltage applied to the anode, as illustrated in FIG. 4a.

The current measured at each voltage at the terminals of the measurement resistor is then plotted against the difference in voltages Δ(AE−B). The curve obtained is linear when voltage applied to the anode is low, for instance less than 2v, corresponding to a difference in voltage between the anode and the body of about 500 mV.

The electrochemical skin conductance, being the slope of the curve, i.e. the ratio between the current measured and the difference in voltages between an active electrode 110 and the body Δ(AE−B), is then computed, as shown in FIG. 4b. This step of computation and plotting is referenced as 202 on FIG. 2.

### Comparison with Control Patients 203

FIG. 4a shows the plot of current against the difference in voltages Δ(AE−B), which in this case corresponds to Va−Vx, for each voltage applied to the anode.

FIG. 4b shows the plot of the electrochemical skin conductance against the difference in voltages Δ(AE−B) for each voltage applied to the anode.

In FIGS. 4a and 4b, the curves with diamond shaped data-points represent measurements of a control test run on a healthy patient, with standard proportions of cholinergic and adrenergic nervous receptors being as follows: 80% of cholinergic nervous receptors and 20% of adrenergic nervous receptors. The curves with square shaped data-points represent a patient with a lower density of adrenergic nervous receptors (curve “Impaired innervation”).

As is visible on FIG. 4b, in the control test run on the healthy patient, the electrochemical skin conductance, which is the slope of the curve of current (FIG. 4a) against the voltage difference Δ(AE−B) between an active electrode 110 and the body, increases with the voltage difference, whereas for a patient with impaired adrenergic nervous receptors, the electrochemical skin conductance is roughly constant with the voltage difference.

In particular, the evolution of the electrochemical skin conductance with the voltage difference between an active electrode 110 and the body varies with the relative proportions of adrenergic and cholinergic nervous receptors.

Thus, in order to assess a relative proportion of adrenergic and cholinergic nervous receptors, one can compute at least two values of the electrochemical skin conductance, for different differences in voltages between the anode and the body, and then compute the difference dESC or the ratio between those values.

FIG. 5 shows the plot of the ratio of electrochemical skin conductance at a given voltage difference Δ(AE−B) between the active electrodes and the body relatively to a voltage difference equal to 300 mV, against the voltage difference.

The curve with square shaped data-points corresponds to an impaired person, and as visible from FIG. 5, this curve is roughly constant. For a control person, the curve increases with the voltage difference between the active electrodes 110 and the body Δ(AE−B).

One can then compare the thus obtained a ratio or difference dESC at two determined values of voltage difference between an active electrode 110 and the body to predetermined thresholds obtained from the application of the same measurements on patients having known relative proportions of adrenergic and cholinergic nerves in order to deduce the proportion from the test subject.

Preferably, among the two values of the electrochemical skin conductance, one value is computed for an intermediate voltage difference between an active electrode 110 and the body, and the other is computed for a higher voltage difference. This allows assessing the evolution of the electrochemical skin conductance relative to voltage with better precision.

Preferably, the intermediate difference voltage elected for the first value of electrochemical skin conductance is chosen from a range of 300 to 500 mV, preferably about 400 mV. The corresponding voltage applied to the anode varies depending on the subjects, but it corresponds roughly to a range of 1.3 to 1.8 V and preferably close to 1.5 V.

The higher voltage difference Δ(AE−B) elected for the second value of electrochemical skin conductance is chosen from 700 mV to 900 mV and preferably about 800 mV, which corresponds rougly to a voltage Va applied to the anode of 3.5 V to 4.5 V and preferably close to 4 V.

It has been found that the most discriminant measurements are those carried out at around 1.5 V and 4 V applied to the anode, or 400 mV and 800 mV of voltage difference Δ(AE-B). Accordingly, the measurement step 202 carried out on the patient can be limited to the measurement of the anode or cathode and body potentials, as well as the current flowing in between, during the application to the anode of two waves of current during 1 second each where the voltages are 1.5 V and 4 V respectively.

The electronic board 120 thus only computes the Δ(AE−B) at the elected voltages, for instance at 1.5 V and 4 V, or at voltages corresponding respectively to 400 mV and 800 mV of voltage difference Δ(AE−B). Similarly the electronic board only computes the electrochemical skin conductance of the patient and the dESC (difference between the electrochemical skin conductance at 1.5 V and 4 V) or ratio at these voltages.

In a preferred embodiment, a sequence of 16 pulses is applied to the electrodes, the voltage applied to the anode at the first pulse being equal to 1 V and, the voltage growing stepwise of 0.2 V until the last pulse of 4V. In that case, a ratio is preferably computed between the electrochemical skin conductance at the second pulse (the applied voltage being of 1.2 V) and at the 14^{th} pulse (applied voltage of 3.6 V). In this example, an appropriate value of the threshold to which the ratio is to be compared ranges from 1.1 to 1.2 and is preferably equal to 1.19 as this ratio has been found to be the most discriminating.

### Disease Diagnostic 204

As mentioned previously, some diseases impact the density of one the two kinds of nervous receptors among the cholinergic and adrenergic ones. Therefore, once the ratio or difference between two values of electrochemical skin conductance has been determined, and that relative proportions of adrenergic and cholinergic nerves have been assessed, a disease can be diagnosed based on these proportions. Of course, other medical or physiological parameters can be taken into account for realizing the diagnostic as well.

For instance, if the method results in determining that the ratio is below a predetermined threshold, the patient probably has a disease that induces imbalance between adrenergic and cholinergic receptors. These diseases and conditions may include but are not limited to trauma, infarction, infection, degenerative nerve disease, malignancy, or post-operative changes including but not limited to Alzheimer's Disease, Parkinson's Disease, Huntington's Chorea, and amyotrophic lateral sclerosis. Another application is to use this imbalance between two kind of receptors in sweat glands as the degree of imbalance of nervous autonomic system in periphery and thus as an indicator of cardiac autonomic system (i.e. central) imbalance.

### Follow-Up of Disease Evolution or Treatment 205

The above described method allows monitoring the evolution of a disease or of its treatment without any invasive examination. The repeated implementation of this method in order to compute relative proportions of adrenergic and cholinergic nervous receptors at different stages of a disease or of a treatment gives the evolution of the relative proportions and thus of the densities of adrenergic and cholinergic receptors. As the adrenergic nervous receptors regenerate faster than the cholinergic nervous receptors, the relative proportions of the two kinds of nervous receptors vary with the regeneration of adrenergic receptors. The efficiency of a treatment can therefore be easily assessed.

### Summary of the Method 200

In summary and without limitation, a method 200 for assessing relative proportions of adrenergic and cholinergic nervous receptors in a patient has been described. The measurement step 201 of the method 200 generally includes contacting an anode and a cathode to different areas of the patient, contacting at least one passive electrode to the patient, and applying voltage pulses to at least one of the anode and the cathode to cause the patient to sweat. The measurement step 201 further includes receiving electrical signals from the anode, cathode, and/or the at least one passive electrode that are representative of the current and voltage potential associated with the anode and the cathode.

The computation and plotting step 202 of the method 200 includes determining electrochemical skin conductance of the patient based, at least in part, on the electrical signals received from the anode, cathode, and/or the at least one passive electrode in the measurement step 201. The computation and plotting step 202 also includes determining relative densities of at least one of cholinergic nervous receptors of the patient and adrenergic nervous receptors of the patient based at least in part on the electrochemical skin conductance of the patient.

The comparison step 203 of the method 200 includes comparing the received signals with reference information representing a person in a control group having a known density of cholinergic and adrenergic nervous receptors. An output of this comparison is also used for determining the relative densities. From the comparison step 203, the method proceeds to the diagnosis step where data from the measurement step 201, the computation and plotting step 202, and the comparison step 203 may be utilized to diagnose the patient as suffering from a disease or disorder.

The method 200 may further include determining a ratio between the current flowing through the anode and the cathode and the voltage difference between the patient and at least one of the anode and the cathode. Further, the method 200 may include automatically changing the voltage pulses applied to at least one of the anode and the cathode in multiple stepwise increments. By way of example and without limitation, the duration of the voltage pulses may range from 0.5 seconds to 2 seconds and the multiple stepwise increments in the voltage pulses may have a step difference ranging from 0.1 V to 0.3 V. Accordingly, the method may call for ten or more stepwise increments in the voltage pulses. The method 200 may also include the optional follow-up set 205 where the progression of the disease or disorder is monitored over time to observe changes in the density of cholinergic and adrenergic nervous receptors in the patient.

One or more of the steps of the method 200 may be performed by an electronic controller 130. The electronic controller 130 thus may have non-transient computer memory storing programmed software instructions for executing various steps of the method.

### Software Carrying Out Steps 201-205

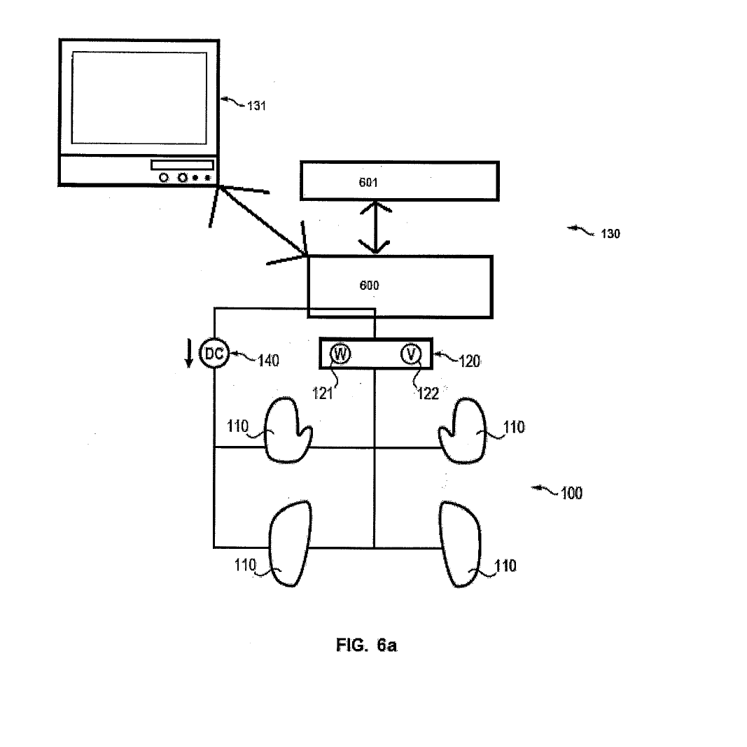

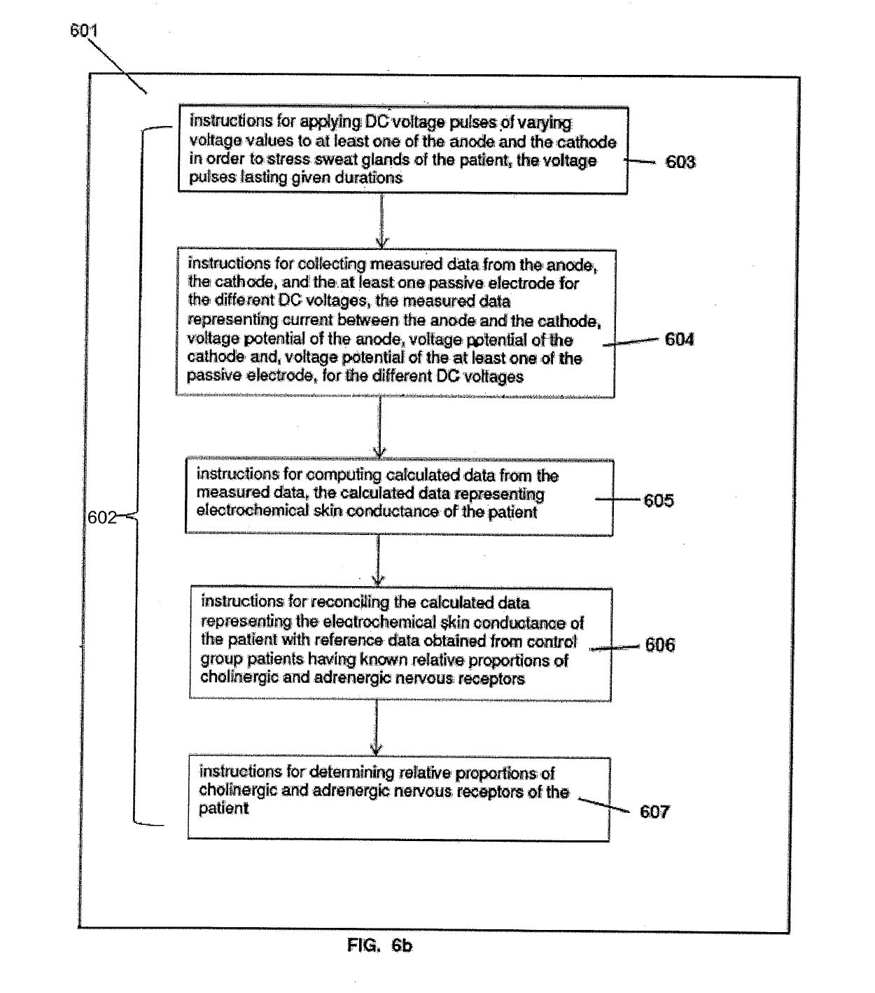

With reference now to FIG. 6a, a computer system 130 for assessing relative proportions of adrenergic and cholinergic nervous receptors in a patient is illustrated. It should be appreciated that such a computer system 130 may be connected to an anode, a cathode, and at least one passive electrode during operation. It should also be appreciated that the four electrodes 110 shown can switch roles of being the anode, cathode, and passive electrode depending on electricity flow such that any of the electrodes 110 may be the anode, the cathode, or the passive electrode at any given time.

The computer system 130 includes non-transient memory 601 and a processor 600 that can access machine readable media stored in the non-transient memory 601. It should be appreciated that the computer system 130 may further include a display 131 for outputting information and one or more inputs for receiving information. By way of example and without limitation, the inputs may include connections to the anode, the cathode, the at least one electrode, a keyboard, and/or a mouse pad.

With reference to FIG. 6b, the machine readable media stored in the non-transient memory 601 may include a plurality of instructions 602. These include instruction 603 that provides for applying DC voltage pulses of varying voltage values to at least one of the anode and the cathode in order to stress sweat glands of the patient wherein the voltage pulses last given durations. Instruction 604 provides for collecting measured data from the anode, the cathode, and the at least one passive electrode for the different DC voltages. The measured data represents the current following between the anode and the cathode, the voltage potential of the anode, the voltage potential of the cathode and, the voltage potential of the at least one of the passive electrode. In accordance with instruction 604, this measured data is collected for each of the different DC voltages that are applied to at least one of the anode and the cathode. Instruction 605 provides for computing calculated data from the measured data wherein the calculated data represents electrochemical skin conductance of the patient.

Instruction 606 provides for reconciling the calculated data representing the electrochemical skin conductance of the patient with reference data obtained from control group patients having known relative proportions of cholinergic and adrenergic nervous receptors. Lastly, instruction 607 provides for determining relative proportions of cholinergic and adrenergic nervous receptors of the patient. In accordance with these instructions 602, the electrochemical skin conductance at a given voltage applied on one of the anode or the cathode is determined as the ratio between the current flowing through the anode and the cathode and the voltage difference between the anode or the cathode and the body. Thus, these instructions allow the computer system to diagnose the patient as suffering from a disease or disorder.

The foregoing description of the embodiments has been provided for the purposes of illustration and description. It is not intended to be exhaustive or limiting. Obviously, many modifications and variations of the present invention are possible in light of the above teachings and may be practiced otherwise than as specifically described while within the scope of the appended claims. These antecedent recitations should be interpreted to cover any combination in which the inventive novelty exercises its utility. It should be appreciated that although steps 201-205 of method 200 and instructions 603-607 are described and illustrated herein in a particular order, these steps and instructions may be performed in a different order without departing from the scope of the present disclosure, except where the order of the steps or instructions is otherwise noted.

What is claimed is:

1. A method for assessing relative proportions of cholinergic and adrenergic nervous receptors of a patient, the method being performed in a system comprising:
an anode and a cathode, intended to be placed on different regions of the patient body,
a plurality of passive electrodes, intended to be placed on different regions of the patient body and connected to a mass with high impedances,
an adjustable DC source, that is controlled in order to feed the anode with a DC current,
the method comprising the following steps:
applying DC voltage pulses of varying voltage values in order to stress sweat glands of the patient, the voltage pulses lasting given durations allowing the stabilization of electrochemical phenomena in the body in the vicinity of the electrodes,
collecting data representative of the current between the anode and the cathode, and of the potential of the anode, the cathode and of at least one passive electrodes, for the different DC voltages,
from said data, computing data representative of the electrochemical skin conductance of the patient,
reconciling said data representative of the electrochemical skin conductance of the patient with reference data obtained in the same conditions on patients having known relative proportions of cholinergic and adrenergic nervous receptors, and determining relative proportions of cholinergic and adrenergic nervous receptors of the patient.

2. A method according to claim 1, wherein the electrochemical skin conductance value at a given voltage applied on the anode is determined as the ratio between the current through the anode and the cathode and the voltage difference between the anode or the cathode and the body.

3. A method according to claim 2, wherein the data representative of the electrochemical skin conductance of the patient comprise the difference and/or ratio between two electrochemical skin conductance values of the patient for an intermediate and a high voltage difference between the anode or the cathode and the body.

4. A method according to claim 3, wherein the intermediate voltage difference between the anode or the cathode and the body ranges from 300 to 500 mV, preferably close to 400 mV, and the high voltage value ranges from 700 to 900 mV, preferably close to 800 mV.

5. A method according to claim 3, wherein the reconciling step comprises determining whether the difference and/or the ratio between the two electrochemical skin conductance values of the patient for intermediate and high voltage difference values between the anode or the cathode and the body is below a given threshold.

6. A method according to claim 5, wherein the reconciling step comprises determining whether the ratio between the two electrochemical skin conductance values of the patient for intermediate and high voltage difference values between the anode or the cathode and the body is below a threshold ranging from 1.1 to 1.2.

7. A method according to claim 1, wherein the duration of the pulses ranges from 0.5 seconds to 2 seconds.

8. A method according to claim 1, wherein the voltage values of the pulses increase and/or decrease stepwise.

9. A method according to claim 8, wherein the step increase or decrease between two successive pulses ranges from 0.1V to 0.3 V.

10. A method according to claim 1, further comprising reconciling the relative proportions of cholinergic and adrenergic nervous receptors of a patient with reference data of patients having the same relative proportions of cholinergic and adrenergic nervous receptors, and identified as suffering or not from a degenerative disease or nervous condition impacting the density of adrenergic or cholinergic nervous receptors, and identifying the patient as suffering or not from said disease.

11. A method for monitoring the evolution of a disease impacting the density of adrenergic or cholinergic nervous receptors, comprising the repeated implementation of the method according to claim 1 on a patient during the monitoring and/or treatment of said disease.

12. A system for assessing relative proportions of adrenergic and cholinergic nervous receptors of a patient, comprising
an anode and a cathode, intended to be placed on different regions of the patient body,
a plurality of passive electrodes, intended to be placed on different regions of the patient body and connected to a mass with high impedances,
an adjustable DC source, that is controlled in order to feed the anode with pulses of a DC current of varying voltage values, for given durations allowing the stabilization of electrochemical phenomena in the body in the vicinity of the electrodes,
a measuring circuit, designed to collect data representative of the current between the anode and the cathode, and of the potentials of the anode, the cathode and of at least a passive electrodes, for the different DC voltages,
wherein the system further comprise a computing circuit, designed to compute data representative of the electrochemical skin conductance of the patient and to reconcile said data with reference data obtained in the same conditions on patients having known relative proportions of adrenergic and cholinergic nervous receptors.

13. A system according to claim 12, wherein the computing circuit is designed to compute electrochemical skin conductance of the patient, being the ratio between the current through the anode and the cathode, and the difference in voltages between the anode or the cathode and the body, for at least two values of voltage differences between the anode or the cathode anode the body.

14. A system according to claim 13, wherein the computing circuit is further designed to compute the ratio and/or the difference between the electrochemical skin conductance computed for two values of voltage differences between the anode or the cathode anode the body, and to compare said ratio or said difference to a given threshold.

15. A system according to claim 14, wherein the computing circuit is further designed to compute the ratio between the electrochemical skin conductance computed for two values of voltage differences between the anode or the cathode anode the body, and to compare said ratio or said difference to a threshold ranging from 1.1 to 1.2.

16. A computer system for assessing relative proportions of adrenergic and cholinergic nervous receptors in a patient using an anode, a cathode, and at least one passive electrode, the computer system comprising:
non-transient memory;
a processor that can access machine readable media stored in the non-transient memory including:
instructions for applying DC voltage pulses of varying voltage values to at least one of the anode and the cathode in order to stress sweat glands of the patient, the voltage pulses lasting given durations;
instructions for collecting measured data from the anode, the cathode, and the at least one passive electrode for the different DC voltages, the measured data representing current between the anode and the cathode, voltage potential of the anode, voltage potential of the cathode and, voltage potential of the at least one of the passive electrode, for the different DC voltages;
instructions for computing calculated data from the measured data, the calculated data representing electrochemical skin conductance of the patient;
instructions for reconciling the calculated data representing the electrochemical skin conductance of the patient with reference data obtained from control group patients having known relative proportions of cholinergic and adrenergic nervous receptors; and
instructions for determining relative proportions of cholinergic and adrenergic nervous receptors of the patient.

17. The computer system according to claim 16, wherein the electrochemical skin conductance value at a given voltage applied on at least one of the anode and the cathode is determined as the ratio between the current flowing through the anode and the cathode and the voltage difference between the body and at least one of the anode and the cathode.

18. A method for assessing relative proportions of adrenergic and cholinergic nervous receptors in a patient, the method comprising:
(a) contacting an anode and a cathode to different areas of the patient;
(b) contacting an electrode to the patient;
(c) applying voltage pulses to at least one of the anode and the cathode to cause the patient to sweat;
(d) receiving electrical signals from at least one of the anode, the cathode, and the electrode that are representative of current and voltage potential associated with the anode and the cathode;
(e) determining electrochemical skin conductance of the patient, based at least in part on the electrical signals received in step (d) with an electronic controller having programmed software instructions stored in nontransient computer memory;
(f) determining with the electronic controller relative densities of at least one of cholinergic nervous receptors of the patient and adrenergic nervous receptors of the patient based at least in part on the electrochemical skin conductance of the patient determined in step (e);
(g) comparing the received signals with reference information representative of a person having a known density of cholinergic and adrenergic nervous receptors, using the electronic controller, and using an output of the comparison for the determining relative densities of step (f);
(h) determining with the electronic controller a ratio between the current flowing through the anode and the cathode, and the voltage difference between the patient and at least one of the anode and the cathode; and
(i) using the electronic controller to automatically change the voltage pulses applied to at least one of the anode and the cathode in multiple stepwise increments.

19. The method according to claim 18, wherein the duration of the voltage pulses ranges from 0.5 seconds to 2 seconds.

20. The method according to claim 19, wherein the multiple stepwise increments in the voltage pulses have a step difference ranging from 0.1 V to 0.3 V.
